SEQUENCE LISTING (58 sequences)

SEQ ID NO: 1 - DNA, 1258 bp, Citrullus lanatus
  source 1..1258: mol_type = genomic DNA
ttaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttga

SEQ ID NO: 2 - DNA, 3146 bp, Citrullus lanatus
  source 1..3146: mol_type = genomic DNA
caaggataattttaaaataatgaagctattatttgaaatatttaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaatcaagtttagttttttccctacaaattatattagtatatattatttactaataatttaactaagttaaaaatatcaagtattctagttctgattcctactaataaacggttaaaagtacctattgattcaaaatcttttaaatttatgggttatgataattttagcactctttcttaattagatagaggaaatgacattatcgagagttttaacacataagcataagctaattggatgatagtatatttagaataggttcggtttaatttctcaagtgtttattaaataaataaaaataagtcattttgaaaaaagttgaagtatttaataaccactcaaaattacttttgaaaaaataaatttatgaaataaacacttacaaataaaccttttaaaaaaattattttgagggtttcaatagttaatctctctctaatttgtatctcgatcaataaaagtatttttaaatacaaatttgaaattattagttcattgtatttaatacttgtccattataataatttgacaccgatggaaacactttgagccaatatataatcaaccaaacaaacttgtatgcaaggttcgaaatattgacggcgatggaaatatcgaggtcctgattttatgaaaatatcgatgtttatgaaaattttgggaaactttatagaaattgatggaaattggttgtaatgagttaataaaactttgattgtggatataattgaccatttttaaacatcattctataaagtaggacaacatttaaatgtatattgtaaatgtcgatgcaagagcataaattttaaaaaaaaaataaaaaattaattacaaaataatataaaacttaggaatattttgaggtgaaattatgtgaaaatttaatgggagaagtatgaaagagattaaaaataaatataggttcttttttgtggtattttttcaaattcaatgaaacttttttgtggaatgaaattttttgatcgaaaataaagctaaaaattgagaaaattttgaaattagaaaaagcagcacattggcaacgcttcattcttcgtcagaaaggagattaacgtctaaaaatatacaattgcgtgtaatcatggatcataaaactaaaaaaccttgaacttagctaacataacaatttctttttttaaaaaatcaaaatttcctcgaaatattctagaaattttctcaagatggaggagaaactaaatcccctggccaaattattttccatcatgcctgtatgtaaaaatacttttgagaataaacataaccacaatttttaatttaaagggctgcacaaaaatagatatagtccagacataatctaaatatttctctaccaaaaaaaatactttttcatgttaattataggtaacacttttaacaaaagggttagtgtattaaagtggtagattataggttggagtaatgtttctccttatcagcctattgaatataaaggcagtaagaagagcgatgaggcgtttggaattgtggaagaggaaagaaacgagaggccccaaatcgctagcctttacaattcactctctaacgctggcgcttgttgagttggtttttgttttgttttgttttgctttgctttagtgtcccctacataaccttccctctccttcttaccttatcctatccttactccttattcccttactcttcacttctcttcttctctctatctctctctctctttcttttaacatttctttatgtaaacaacccaacccatctttacataaccttcttcattttccctcaaagtttcccaatttctcaattggatttaacccttttgcattgccataatccaaact

SEQ ID NO: 3 - DNA, 4404 bp, Citrullus lanatus
  source 1..4404: mol_type = genomic DNA
caaggataattttaaaataatgaagctattatttgaaatatttaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaatcaagtttagttttttccctacaaattatattagtatatattatttactaataatttaactaagttaaaaatatcaagtattctagttctgattcctactaataaacggttaaaagtacctattgattcaaaatcttttaaatttatgggttatgataattttagcactctttcttaattagatagaggaaatgacattatcgagagttttaacacataagcataagctaattggatgatagtatatttagaataggttcggtttaatttctcaagtgtttattaaataaataaaaataagtcattttgaaaaaagttgaagtatttaataaccactcaaaattacttttgaaaaaataaatttatgaaataaacacttacaaataaaccttttaaaaaaattattttgagggtttcaatagttaatctctctctaatttgtatctcgatcaataaaagtatttttaaatacaaatttgaaattattagttcattgtatttaatacttgtccattataataatttgacaccgatggaaacactttgagccaatatataatcaaccaaacaaacttgtatgcaaggttcgaaatattgacggcgatggaaatatcgaggtcctgattttatgaaaatatcgatgtttatgaaaattttgggaaactttatagaaattgatggaaattggttgtaatgagttaataaaactttgattgtggatataattgaccatttttaaacatcattctataaagtaggacaacatttaaatgtatattgtaaatgtcgatgcaagagcataaattttaaaaaaaaaataaaaaattaattacaaaataatataaaacttaggaatattttgaggtgaaattatgtgaaaatttaatgggagaagtatgaaagagattaaaaataaatataggttcttttttgtggtattttttcaaattcaatgaaacttttttgtggaatgaaattttttgatcgaaaataaagctaaaaattgagaaaattttgaaattagaaaaagcagcacattggcaacgcttcattcttcgtcagaaaggagattaacgtctaaaaatatacaattgcgtgtaatcatggatcataaaactaaaaaaccttgaacttagctaacataacaatttctttttttaaaaaatcaaaatttcctcgaaatattctagaaattttctcaagatggaggagaaactaaatcccctggccaaattattttccatcatgcctgtatgtaaaaatacttttgagaataaacataaccacaatttttaatttaaagggctgcacaaaaatagatatagtccagacataatctaaatatttctctaccaaaaaaaatactttttcatgttaattataggtaacacttttaacaaaagggttagtgtattaaagtggtagattataggttggagtaatgtttctccttatcagcctattgaatataaaggcagtaagaagagcgatgaggcgtttggaattgtggaagaggaaagaaacgagaggccccaaatcgctagcctttacaattcactctctaacgctggcgcttgttgagttggtttttgttttgttttgttttgctttgctttagtgtcccctacataaccttccctctccttcttaccttatcctatccttactccttattcccttactcttcacttctcttcttctctctatctctctctctctttcttttaacatttctttatgtaaacaacccaacccatctttacataaccttcttcattttccctcaaagtttcccaatttctcaattggatttaacccttttgcattgccataatccaaact

SEQ ID NO: 4 - DNA, 5662 bp, Citrullus lanatus
  source 1..5662: mol_type = genomic DNA
caaggataattttaaaataatgaagctattatttgaaatatttaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaatcaagtttagttttttccctacaaattatattagtatatattatttactaataatttaactaagttaaaaatatcaagtattctagttctgattcctactaataaacggttaaaagtacctattgattcaaaatcttttaaatttatgggttatgataattttagcactctttcttaattagatagaggaaatgacattatcgagagttttaacacataagcataagctaattggatgatagtatatttagaataggttcggtttaatttctcaagtgtttattaaataaataaaaataagtcattttgaaaaaagttgaagtatttaataaccactcaaaattacttttgaaaaaataaatttatgaaataaacacttacaaataaaccttttaaaaaaattattttgagggtttcaatagttaatctctctctaatttgtatctcgatcaataaaagtatttttaaatacaaatttgaaattattagttcattgtatttaatacttgtccattataataatttgacaccgatggaaacactttgagccaatatataatcaaccaaacaaacttgtatgcaaggttcgaaatattgacggcgatggaaatatcgaggtcctgattttatgaaaatatcgatgtttatgaaaattttgggaaactttatagaaattgatggaaattggttgtaatgagttaataaaactttgattgtggatataattgaccatttttaaacatcattctataaagtaggacaacatttaaatgtatattgtaaatgtcgatgcaagagcataaattttaaaaaaaaaataaaaaattaattacaaaataatataaaacttaggaatattttgaggtgaaattatgtgaaaatttaatgggagaagtatgaaagagattaaaaataaatataggttcttttttgtggtattttttcaaattcaatgaaacttttttgtggaatgaaattttttgatcgaaaataaagctaaaaattgagaaaattttgaaattagaaaaagcagcacattggcaacgcttcattcttcgtcagaaaggagattaacgtctaaaaatatacaattgcgtgtaatcatggatcataaaactaaaaaaccttgaacttagctaacataacaatttctttttttaaaaaatcaaaatttcctcgaaatattctagaaattttctcaagatggaggagaaactaaatcccctggccaaattattttccatcatgcctgtatgtaaaaatacttttgagaataaacataaccacaatttttaatttaaagggctgcacaaaaatagatatagtccagacataatctaaatatttctctaccaaaaaaaatactttttcatgttaattataggtaacacttttaacaaaagggttagtgtattaaagtggtagattataggttggagtaatgtttctccttatcagcctattgaatataaaggcagtaagaagagcgatgaggcgtttggaattgtggaagaggaaagaaacgagaggccccaaatcgctagcctttacaattcactctctaacgctggcgcttgttgagttggtttttgttttgttttgttttgctttgctttagtgtcccctacataaccttccctctccttcttaccttatcctatccttactccttattcccttactcttcacttctcttcttctctctatctctctctctctttcttttaacatttctttatgtaaacaacccaacccatctttacataaccttcttcattttccctcaaagtttcccaatttctcaattggatttaacccttttgcattgccataatccaaact

SEQ ID NO: 5 - DNA, 6920 bp, Citrullus lanatus
  source 1..6920: mol_type = genomic DNA
caaggataattttaaaataatgaagctattatttgaaatatttaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaaatacacaacaactaaattgacgtaaacatcacattcttaaaggctcaagccgggtctaaattaaaagcatcaccaaaaacttccaaatcgccattaaaaaaagaaaacaaaaagaagaaaaatgaatagaaatatcacaacaattagggtaaaaagaaagctattttataaacatcttttctttaaatggtgtaacgttgaattattgaatgaaagaagagagaaagggatcttatcatgtggagattgctcaagtagtttgtccattggttgaacactccaaaatgggaggttctcgtaacactctcatctatcctttttccatttacctaaccaaatctaaagcaagaggttagagtgggaaaaatatgggttatcgagaacgggccaaaattgaaataatgctaatttttattaaataatttgtcaactgaaataaagggcaccaaccttgagtagcaacattttcaaagaattgcaaatatagctgttagaatgccttgaatttgttatctgacgcttcgaaggaccaagtatagcccctgaaacctaagttgatttcatatttggcatatataagcatctataattatttacgattatgaccctaagatttagagaaatgcacaatataaactttctaactttagaggcgccactattctgtaacattatttaataaaattgttctttctctacctcatgaatgtagctaatacactcttagtaaaccatataaatctttgttcgattctcaactattttacatttttctttattctttatttgtcgattttgtaacactaacaaaatctatcaatggtagattctatcgttgatagactcctatgagtgatagactctgagagcttgatctaaattttgttatatttgcaaaaaattttgcattgtgctatatctattaatactttgggtctaattacggtatttgtaactgcctctcttacatatgcaattttattcttttgaacatctataattatgtctaatccacaaactaatattcaataattttgccaaaattcataaatttcaaaaaaaaaaaaaaaaaaaaaaaaaagccttgaaggcctttttatttaacaaatttatagcatgcataagttttaaccatatttggaagaacgtgtgaaagaaggtaagattgcaacaccttttaaatcttaaaattaaaattataaaatacttatctaagtttgataggtttggattaaaaatttgatgcaacttgattaaatcaagtttagttttttccctacaaattatattagtatatattatttactaataatttaactaagttaaaaatatcaagtattctagttctgattcctactaataaacggttaaaagtacctattgattcaaaatcttttaaatttatgggttatgataattttagcactctttcttaattagatagaggaaatgacattatcgagagttttaacacataagcataagctaattggatgatagtatatttagaataggttcggtttaatttctcaagtgtttattaaataaataaaaataagtcattttgaaaaaagttgaagtatttaataaccactcaaaattacttttgaaaaaataaatttatgaaataaacacttacaaataaaccttttaaaaaaattattttgagggtttcaatagttaatctctctctaatttgtatctcgatcaataaaagtatttttaaatacaaatttgaaattattagttcattgtatttaatacttgtccattataataatttgacaccgatggaaacactttgagccaatatataatcaaccaaacaaacttgtatgcaaggttcgaaatattgacggcgatggaaatatcgaggtcctgattttatgaaaatatcgatgtttatgaaaattttgggaaactttatagaaattgatggaaattggttgtaatgagttaataaaactttgattgtggatataattgaccatttttaaacatcattctataaagtaggacaacatttaaatgtatattgtaaatgtcgatgcaagagcataaattttaaaaaaaaaataaaaaattaattacaaaataatataaaacttaggaatattttgaggtgaaattatgtgaaaatttaatgggagaagtatgaaagagattaaaaataaatataggttcttttttgtggtattttttcaaattcaatgaaacttttttgtggaatgaaattttttgatcgaaaataaagctaaaaattgagaaaattttgaaattagaaaaagcagcacattggcaacgcttcattcttcgtcagaaaggagattaacgtctaaaaatatacaattgcgtgtaatcatggatcataaaactaaaaaaccttgaacttagctaacataacaatttctttttttaaaaaatcaaaatttcctcgaaatattctagaaattttctcaagatggaggagaaactaaatcccctggccaaattattttccatcatgcctgtatgtaaaaatacttttgagaataaacataaccacaatttttaatttaaagggctgcacaaaaatagatatagtccagacataatctaaatatttctctaccaaaaaaaatactttttcatgttaattataggtaacacttttaacaaaagggttagtgtattaaagtggtagattataggttggagtaatgtttctccttatcagcctattgaatataaaggcagtaagaagagcgatgaggcgtttggaattgtggaagaggaaagaaacgagaggccccaaatcgctagcctttacaattcactctctaacgctggcgcttgttgagttggtttttgttttgttttgttttgctttgctttagtgtcccctacataaccttccctctccttcttaccttatcctatccttactccttattcccttactcttcacttctcttcttctctctatctctctctctctttcttttaacatttctttatgtaaacaacccaacccatctttacataaccttcttcattttccctcaaagtttcccaatttctcaattggatttaacccttttgcattgccataatccaaact

SEQ ID NO: 6 - DNA, 22 bp, Synthetic construct
  source 1..22: mol_type = other DNA
caaggataattttaaaataatg

SEQ ID NO: 7 - DNA, 23 bp, Synthetic construct
  source 1..23: mol_type = other DNA
atatactaatataatttgtaggg

SEQ ID NO: 8 - DNA, 24 bp, Synthetic construct
  source 1..24: mol_type = other DNA
gtaaagatgggttgggttgtttac

SEQ ID NO: 9 - DNA, 47 bp, Synthetic construct
  source 1..47: mol_type = other DNA
gaaggtgaccaagttcatgctaagccatcgaattcgacactcaaaat

SEQ ID NO: 10 - DNA, 47 bp, Synthetic construct
  source 1..47: mol_type = other DNA
gaaggtcggagtcaacggattaagccatcgaattcgacactcaaaaa

SEQ ID NO: 11 - DNA, 27 bp, Synthetic construct
  source 1..27: mol_type = other DNA
gctgaatttttctggggttcgtctgtt

SEQ ID NO: 12 - DNA, 50 bp, Synthetic construct
  source 1..50: mol_type = other DNA
gaaggtgaccaagttcatgctaatcgtgtgaaacttacactcatttaaca

SEQ ID NO: 13 - DNA, 47 bp, Synthetic construct
  source 1..47: mol_type = other DNA
gaaggtcggagtcaacggattcgtgtgaaacttacactcatttaacg

SEQ ID NO: 14 - DNA, 34 bp, Synthetic construct
  source 1..34: mol_type = other DNA
gcatcactaacatttgtcatttgtcgaataatta

SEQ ID NO: 15 - DNA, 48 bp, Synthetic construct
  source 1..48: mol_type = other DNA
gaaggtgaccaagttcatgcttattgcattagggatttcaacccttac

SEQ ID NO: 16 - DNA, 49 bp, Synthetic construct
  source 1..49: mol_type = other DNA
gaaggtcggagtcaacggattctattgcattagggatttcaacccttat

SEQ ID NO: 17 - DNA, 30 bp, Synthetic construct
  source 1..30: mol_type = other DNA
gtgctactccactcataaccaattagtttt

SEQ ID NO: 18 - DNA, 30 bp, Synthetic construct
  source 1..30: mol_type = other DNA
tagtcctcattcgtgaattttaaattttgt

SEQ ID NO: 19 - DNA, 28 bp, Synthetic construct
  source 1..28: mol_type = other DNA
gtcctcattcgtgaattttaaattttgc

SEQ ID NO: 20 - DNA, 44 bp, Synthetic construct
  source 1..44: mol_type = other DNA
gaatttctaccatatagactatataattattaattttaaaatat

SEQ ID NO: 21 - DNA, 50 bp, Synthetic construct
  source 1..50: mol_type = other DNA
gaaggtgaccaagttcatgcttttatacaagactccatgaataatctcag

SEQ ID NO: 22 - DNA, 51 bp, Synthetic construct
  source 1..51: mol_type = other DNA
gaaggtcggagtcaacggattatttatacaagactccatgaataatctcat

SEQ ID NO: 23 - DNA, 31 bp, Synthetic construct
  source 1..31: mol_type = other DNA
agggtgtgatttcattaaatacaccattaag

SEQ ID NO: 24 - DNA, 21 bp, Synthetic construct
  source 1..21: mol_type = other DNA
gcaacccctcccaaccatctt

SEQ ID NO: 25 - DNA, 20 bp, Synthetic construct
  source 1..20: mol_type = other DNA
caacccctcccaaccatctc

SEQ ID NO: 26 - DNA, 28 bp, Synthetic construct
  source 1..28: mol_type = other DNA
catatcggtccctttatcaattctttcc

SEQ ID NO: 27 - DNA, 152 bp, Synthetic construct
  source 1..152: mol_type = other DNA
ttaaagaatacacaaaactgaatagtagctgtcagtacttttcttcatttagaaaccaaaagagacatataaagagaaggaggagcaatgccaatgaccataactaagagaacaaaacaaatgagagaaaatgcatatcaatcaaattaccc

SEQ ID NO: 28 - DNA, 43 bp, Synthetic construct
  source 1..43: mol_type = other DNA
gaaggtgaccaagttcatgctgtcattggcattgctcctcctc

SEQ ID NO: 29 - DNA, 43 bp, Synthetic construct
  source 1..43: mol_type = other DNA
gaaggtcggagtcaacggattgtcattggcattgctcctcctt

SEQ ID NO: 30 - DNA, 152 bp, Synthetic construct
  source 1..152: mol_type = other DNA
aagggttgagtgttaagcttctcgggagtcaccgtaattatgttgtaattccagataagtattttattgtaattgcgttgacattattaatagtgaagttatatttcctagattgctgctccctagacataggtgatattattgcaccgaac

SEQ ID NO: 31 - DNA, 53 bp, Synthetic construct
  source 1..53: mol_type = other DNA
gaaggtgaccaagttcatgctgtaattccagataagtattttattgtaattgc

SEQ ID NO: 32 - DNA, 53 bp, Synthetic construct
  source 1..53: mol_type = other DNA
gaaggtcggagtcaacggattgtaattccagataagtattttattgtaattgg

SEQ ID NO: 33 - DNA, 152 bp, Synthetic construct
  source 1..152: mol_type = other DNA
gagttaaacagaagcaaaatattgaaaatgaaaattaggttaccttgaaagaagggagggtaaatgaaggtgggggaggaagccaagcattaaagctaccaaagcaacacaacattattttaaccctacaattccttcattcaaatgtcgtt

SEQ ID NO: 34 - DNA, 44 bp, Synthetic construct
  source 1..44: mol_type = other DNA
gaaggtgaccaagttcatgctgtagctttaatgcttggcttccc

SEQ ID NO: 35 - DNA, 45 bp, Synthetic construct
  source 1..45: mol_type = other DNA
gaaggtcggagtcaacggattggtagctttaatgcttggcttcct

SEQ ID NO: 36 - DNA, 152 bp, Synthetic construct
  source 1..152: mol_type = other DNA
aaattagagcctcaatttggcaccttataatctcgaatgctagaaacaatcatgatattaacgagagccacacctagactaaaatgattcttgacactataaatagcctcattcaacaactccgaatgagagagtaagtaactttcaaaaga

SEQ ID NO: 37 - DNA, 46 bp, Synthetic construct
  source 1..46: mol_type = other DNA
gaaggtgaccaagttcatgctatgatattaacgagagccacaccta

SEQ ID NO: 38 - DNA, 44 bp, Synthetic construct
  source 1..44: mol_type = other DNA
gaaggtcggagtcaacggattgatattaacgagagccacacctg

SEQ ID NO: 39 - DNA, 43 bp, Synthetic construct
  source 1..43: mol_type = other DNA
gaaggtgaccaagttcatgctcctccatccaaatggccttctc

SEQ ID NO: 40 - DNA, 44 bp, Synthetic construct
  source 1..44: mol_type = other DNA
gaaggtcggagtcaacggattacctccatccaaatggccttcta

SEQ ID NO: 41 - DNA, 30 bp, Synthetic construct
  source 1..30: mol_type = other DNA
gttttgtattggggatttgcggtatagaaa

SEQ ID NO: 42 - DNA, 43 bp, Synthetic construct
  source 1..43: mol_type = other DNA
gaaggtgaccaagttcatgcttaagttggcaagcttaggccgc

SEQ ID NO: 43 - DNA, 46 bp, Synthetic construct
  source 1..46: mol_type = other DNA
gaaggtcggagtcaacggatttattaagttggcaagcttaggccga

SEQ ID NO: 44 - DNA, 34 bp, Synthetic construct
  source 1..34: mol_type = other DNA
atttgtttgagattatccttttatctacatggat

SEQ ID NO: 45 - DNA, 48 bp, Synthetic construct
  source 1..48: mol_type = other DNA
gaaggtgaccaagttcatgctccaatctttggtatttcaactgcaaca

SEQ ID NO: 46 - DNA, 47 bp, Synthetic construct
  source 1..47: mol_type = other DNA
gaaggtcggagtcaacggattcaatctttggtatttcaactgcaacg

SEQ ID NO: 47 - DNA, 30 bp, Synthetic construct
  source 1..30: mol_type = other DNA
cagagttcgaatctttgtgaacatacgaaa

SEQ ID NO: 48 - DNA, 43 bp, Synthetic construct
  source 1..43: mol_type = other DNA
gaaggtgaccaagttcatgctcctcctccaacgctttccaaca

SEQ ID NO: 49 - DNA, 42 bp, Synthetic construct
  source 1..42: mol_type = other DNA
gaaggtcggagtcaacggattctcctccaacgctttccaacc

SEQ ID NO: 50 - DNA, 26 bp, Synthetic construct
  source 1..26: mol_type = other DNA
gtttttgacgcatggcggttggaatt

SEQ ID NO: 51 - DNA, 23 bp, Synthetic construct
  source 1..23: mol_type = other DNA
atggctcccaaagctggaaaaac

SEQ ID NO: 52 - DNA, 22 bp, Synthetic construct
  source 1..22: mol_type = other DNA
tcaacttgaaacctcaacaatc

SEQ ID NO: 53 - DNA, 21 bp, Synthetic construct
  source 1..21: mol_type = other DNA
cagtaggtgcatcatctccag

SEQ ID NO: 54 - DNA, 21 bp, Synthetic construct
  source 1..21: mol_type = other DNA
caatctcagcttcattgtcgc

SEQ ID NO: 55 - DNA, 24 bp, Synthetic construct
  source 1..24: mol_type = other DNA
cctacaactcaattatgaagtgtg

SEQ ID NO: 56 - DNA, 24 bp, Synthetic construct
  source 1..24: mol_type = other DNA
gaaatccacatctgctggaaggtg

SEQ ID NO: 57 - DNA, 23 bp, Synthetic construct
  source 1..23: mol_type = other DNA
gaagttcatttcatttggagagg

SEQ ID NO: 58 - DNA, 21 bp, Synthetic construct
  source 1..21: mol_type = other DNA
acttgaaacctcaacaatctc

(19) United States
(12) Patent Application Publication
(10) Pub. No.: US 2026/0117321 A1
(43) Pub. Date: Apr. 30, 2026

(54) APPLICATION OF CLFCI GENE AND ITS TANDEM REPEAT SEQUENCES IN PROMOTER REGION IN IDENTIFYING WATERMELON FLESH COLOR INTENSITY

(71) Applicant: BEIJING ACADEMY OF AGRICULTURE AND FORESTRY SCIENCES, Beijing (CN)

(72) Inventors: Yong XU, Beijing (CN); Jie ZHANG, Beijing (CN); Haiying ZHANG, Beijing (CN); Guoyi GONG, Beijing (CN); Yi REN, Beijing (CN); Maoying LI, Beijing (CN); Yongtao YU, Beijing (CN); Jinfang WANG, Beijing (CN); Shengjin LIAO, Beijing (CN)

(21) Appl. No.: 19/162,984

(22) PCT Filed: Apr. 3, 2025
(86) PCT No.: PCT/CN2025/087337

§ 371 Date: Sep. 8, 2025

(30) Foreign Application Priority Data
Apr. 30, 2024 (CN) 202410539843.2

(51) Int. Cl. C12Q 1/6895

(52) U.S. Cl. CPC C12Q 1/6895; C12Q 2600/13; C12Q 2600/156

(57) ABSTRACT

The present invention belongs to the field of biotechnology and provides the application of the watermelon flesh color intensity control gene CIFCI gene and its tandem repeat sequences in promoter region in the identification of watermelon flesh color intensity. Based on the presence of the tandem repeat region in the promoter of the CIFCI gene in watermelon varieties, the present invention designs a PCR marker for convenient detection, which can quickly detect the possible flesh color phenotypes in the hybrid offspring with light and deep flesh colors at the seedling stage. The application of this gene marker in marker-assisted breeding can improve the quality of watermelon fruits.

### TECHNICAL FIELD

The present invention belongs to the field of biotechnology, and particularly relates to the application of the CIFCI gene controlling watermelon flesh color intensity and its tandem repeat sequences in promoter region in identifying watermelon flesh color intensity.

### BACKGROUND

Watermelon (Citrullus lanatus) is one of the world's top ten fruits and an important economic crop widely cultivated domestically and internationally. China is the largest producer and consumer of watermelon globally. Watermelon flesh exhibits diverse colors, which can be broadly classified into five types: red, pink, orange-red, yellow, and white across different germplasm resources. The primary reason for these color variations lies in differences in pigment content and composition.

During the breeding of cultivated watermelon varieties, flesh color has undergone a process of improvement from light to deep. However, the key gene(s) controlling flesh color intensity in cultivated watermelon remain unclear. Compared to watermelons with different flesh colors (red, orange, yellow, white), varieties with the same base color but varying intensities are more common in watermelon resources. Significant differences exist in carotenoid content among varieties with different flesh color intensities. For example, in the watermelon variety AU-Sweet Scarlet with red or deep red flesh, the content of lycopene is higher than 50 mg/1000 g fresh weight, while in the pink-fleshed watermelon variety Angeleno Black Seeded, the content of lycopene is 30 mg/1000 g fresh weight. This indicates substantial nutritional differences based on flesh color intensity. It is also difficult to obtain offspring with flesh color deeper than both parents in breeding. Therefore, there is an urgent need to understand the internal molecular mechanisms that controls differences in carotenoid accumulation capacity in watermelon flesh, clarify the key genes (Flesh color intensity, FCI) controlling watermelon flesh color intensity, providing a solid foundation for molecular improvement of watermelons.

### SUMMARY OF THE INVENTION

The inventors of the present invention conducted fine mapping on segregating populations derived from crosses between watermelon inbred lines with different flesh color intensities (deep red/light red; deep yellow/light yellow), and obtained the gene controlling watermelon flesh color intensity (Flesh color intensity CIFCI gene), and discovered that the tandem repeats in the promoter region of this gene in varieties with deep flesh enhanced the expression of the gene, leading to a deeper flesh color.

The present invention provides the application of the watermelon CIFCI gene in controlling watermelon flesh color intensity.

The present invention also provides a method for deepening watermelon flesh color, which includes the operation of overexpression of the CIFCI gene in watermelon plants.

The present invention also provides a tandem repeat sequence related to the control of watermelon flesh color intensity. The tandem repeat sequence is located in the promoter region of the watermelon CIFCI gene, having a length of 1258 bp, with a nucleotide sequence composition as shown in SEQ ID NO: 1.

The present invention also provides a method for identifying watermelon flesh color intensity, which includes the step of detecting the number of said tandem repeat sequences in the promoter region of the CIFCI gene in the watermelon to be tested; wherein said tandem repeat sequence has a length of 1258 bp, with a nucleotide sequence composition as shown in SEQ ID NO: 1; wherein:
When 1 of the tandem repeat sequences is detected, the flesh color of the watermelon to be tested is light;
When 2 of the tandem repeat sequences are detected, the flesh color of the watermelon to be tested is medium or deep;
When 3 or more of the tandem repeat sequences are detected, the flesh color of the watermelon to be tested is deep.

The present invention reveals for the first time the key gene controlling watermelon flesh color intensity and uncover the molecular basis for the continuous deepening of flesh color in cultivated varieties through artificial selection. Based on the presence of tandem repeat regions in the CIFCI gene promoter among watermelon varieties, the invention designs convenient PCR markers for rapid detection of potential flesh color phenotypes in hybrid offspring at the seedling stage. The application of this gene marker in assisted breeding can improve watermelon fruit quality. The invention elucidates the molecular mechanism underlying watermelon flesh color deepening and provides a target gene for creating high-quality watermelon varieties with higher carotenoid content.

### DESCRIPTION OF THE DRAWINGS

FIG. 1: Representative fruit cross-section images of the parental and offspring populations for the mapping of the watermelon CIFCI gene.

FIG. 2: Fine mapping diagram of the watermelon CIFCI gene.

FIG. 3: PCR detection gel image showing CIFCI promoter length in randomly detected different watermelon materials.

FIG. 4: Fruit cross-section images of four representative watermelon materials.

FIG. 5: Analysis chart of four types of CIFCI gene promoter structures existing in different watermelon materials.

FIG. 6: Analysis chart of relative expression levels of the CIFCI gene in different organs of JLM.

FIG. 7: Analysis chart of relative expression levels of the CIFCI gene in fruits of materials with different flesh colors.

FIG. 8: Flesh color phenotype chart of the offspring from CIFCI overexpression transgenic experiments.

### DETAILED DESCRIPTION OF THE INVENTION

To make the technical solutions, objectives, and advantages of the present invention clearer, the invention is further described in detail below through specific embodiments. It should be understood that the specific embodiments described herein are intended only to illustrate and explain the invention, not to limit it.

The first aspect of the present invention provides the application of the watermelon CIFCI gene in controlling watermelon flesh color intensity.

The watermelon CIFCI gene, also referred to as the watermelon flesh color intensity control gene, is registered as Cla97C06G121890 (with a full length of 13908 bp) in the Cucurbit Genomics Database and can be queried at http://cucurbitgenomics.org/v2/feature/gene/Cla97C06G121890.

The second aspect of the present invention provides a method for deepening the color of watermelon flesh, which includes the operation of overexpression of the CIFCI gene in watermelon plants.

According to the second aspect of the present invention, the operation of overexpression of the CIFCI gene in watermelon plants includes the operation of constructing an overexpression vector of CIFCI using the pYBA1302 vector, and then transferring the overexpression vector into watermelon plants.

The third aspect of the present invention provides a tandem repeat sequence related to watermelon flesh color intensity control, said tandem repeat sequence is located in the promoter region of the watermelon flesh color intensity control gene (i.e., the CIFCI gene), having a length of 1258 bp, with a nucleotide sequence composition as shown in SEQ ID NO: 1.

The fourth aspect of the present invention provides the application of the tandem repeat sequence in the promoter region of the watermelon flesh color intensity control gene CIFCI gene provided in the third aspect of the present invention in the identification of watermelon flesh color intensity.

The fifth aspect of the present invention provides a method for identifying watermelon flesh color intensity, which includes the step of detecting the number of tandem repeat sequences in the promoter region of the CIFCI gene of the watermelon to be tested; wherein said tandem repeat sequence has a length of 1258 bp, with a nucleotide sequence composition as shown in SEQ ID NO: 1.

### According to the Fifth Aspect of the Present Invention, Wherein:

When 1 of the tandem repeat sequences is detected, the flesh color of the watermelon to be tested is light;
When 2 of the tandem repeat sequences are detected, the flesh color of the watermelon to be tested is medium or deep;
When 3 or more of the tandem repeat sequences are detected, the flesh color of the watermelon to be tested is deep.

Through resequencing and PCR analysis, the inventors of the present invention discovered structural differences in the promoter region of the watermelon CIFCI gene between the parents with deep and light flesh color. This difference is caused by the fact that the 1,258 bp sequence from −1,848 bp to −3,105 bp before the ATG of the CIFCI gene promoter in light-fleshed watermelons (JX-2, Cream S) is tandemly repeated three times in deep-fleshed varieties (Ming 58, JLM). Subsequent detection across watermelon materials with different flesh colors revealed that this sequence is repeated 1-4 times in various tested varieties, and the number of repeats is related to the depth of the flesh color.

For example, the watermelon variety 97103 (red flesh, light color) and the watermelon variety Cream of Saskatchewan (Cream S, light yellow flesh, light color) have 1 tandem repeat sequence in the CIFCI gene promoter region; specifically, the sequence from −1 bp to −3,146 bp before the ATG of the CIFCI gene promoter in the watermelon variety 97103 is composed of the nucleotide sequence as shown in SEQ ID NO: 2, and it can be found that there is 1 such tandem repeat sequence.

The watermelon variety Xin Hongbao (XHB, red flesh, normal red, medium color), has 2 of the tandem repeat sequences in the promoter region of its CIFCI gene; specifically, the sequence from −1 bp to −4404 bp before the ATG of the CIFCI gene promoter of Xin Hongbao has the nucleotide sequence composition as shown in SEQ ID NO: 3, and it can be found that there are 2 of the tandem repeat sequences.

The watermelon variety Ming 58 (deep red flesh, deep color), and the watermelon variety JLM (deep yellow flesh, deep color), have 3 of the tandem repeat sequences in the promoter region of their CIFCI gene; specifically, the sequence from −1 bp to −5662 bp before the ATG of the CIFCI gene promoter of the watermelon varieties Ming 58 and JLM has the nucleotide sequence composition as shown in SEQ ID NO: 4, and it can be found that there are 3 of the tandem repeat sequences.

The watermelon variety GS89 (Hei Beng Jin, or GS89-HBJ, orange flesh, deep color) has 4 of the tandem repeat sequences in the promoter region of its CIFCI gene. The sequence from −1 bp to −6920 bp before the ATG of the CIFCI gene promoter has the nucleotide sequence composition as shown in SEQ ID NO: 5, and it can be found that there are 4 of the described tandem repeat sequences.

According to the fifth aspect of the present invention, the method for identifying watermelon flesh color intensity includes using the genomic DNA of the watermelon to be tested as a template and performing PCR amplification with the following primer:

Upstream primer:
(SEQ ID NO: 6)
5′-CAAGGATAATTTTAAAATAATG-3′;
Downstream primer:
(SEQ ID NO: 7)
5′-ATATACTAATATAATTTGTAGGG-3′;

If the amplified fragment contains 1 tandem repeat sequence shown in SEQ ID NO: 1, the flesh color of the watermelon to be tested is light; if the amplified fragment contains 2 tandem repeat sequences shown in SEQ ID NO: 1, the flesh color of the watermelon to be tested is medium or deep; if the amplified fragment contains 3 or more tandem repeat sequences shown in SEQ ID NO: 1, the flesh color of the watermelon to be tested is deep.

For example, for the watermelon varieties 97103 and Cream of Saskatchewan, a 1343 bp fragment can be amplified. After sequencing, it was found that this fragment contains 1 tandem repeat sequence of 1258 bp in length as shown in SEQ ID NO: 1. The sequence composition of this 1343 bp fragment is shown from positions 1-1343 of SEQ ID NO: 2 in the Sequence Listing.

For the watermelon variety Xin Hongbao (XHB), a 2601 bp fragment can be amplified. After sequencing, it was found that this fragment contains 2 tandem repeat sequences of 1258 bp in length as shown in SEQ ID NO: 1. The sequence composition of this 2601 bp fragment is shown from positions 1-2601 of SEQ ID NO: 3 in the Sequence Listing.

For the watermelon varieties Ming 58 and JLM, a 3859 bp fragment can be amplified. After sequencing, it was found that this fragment contains 3 tandem repeat sequences of 1258 bp in length as shown in SEQ ID NO: 1. The sequence composition of this 3859 bp fragment is shown from positions 1-3859 of SEQ ID NO: 4 in the Sequence Listing.

For the watermelon variety GS89, a 5117 bp fragment can be amplified. After sequencing, it is found that this fragment contains 4 tandem repeat sequences with a length of 1258 bp as shown in SEQ ID NO: 1. The sequence composition of this 5117 bp fragment is shown as positions 1-5117 of SEQ ID NO: 5 in the Sequence Listing.

According to the fifth aspect of the present invention, the method for identifying watermelon flesh color intensity includes using the genomic DNA of the watermelon to be tested as a template and performing PCR amplification with the following primers:

Upstream sequence:
(SEQ ID NO: 6)
5′-CAAGGATAATTTTAAAATAATG-3′;
Downstream sequence:
(SEQ ID NO: 8)
5′-GTAAAGATGGGTTGGGTTGTTTAC-3′;

If the amplified fragment contains 1 tandem repeat sequence as shown in SEQ ID NO: 1, the flesh color of the watermelon to be tested is light; if the amplified fragment contains 2 tandem repeat sequences as shown in SEQ ID NO: 1, the flesh color of the watermelon to be tested is medium or deep; if the amplified fragment contains 3 or more tandem repeat sequences as shown in SEQ ID NO: 1, the flesh color of the watermelon to be tested is deep.

Using the primer combination of the above-mentioned SEQ ID NO: 6 and SEQ ID NO: 8, the promoter region of the CIFCI gene can be amplified. Then, the number of tandem repeat sequences shown in SEQ ID NO: 1 can be determined by the size of the amplification product. For example, the 3146 bp fragment (the sequence composition shown in SEQ ID NO: 2) contains 1 tandem repeat sequence shown in SEQ ID NO: 1; the 4404 bp fragment (the sequence composition shown in SEQ ID NO: 3) contains 2 tandem repeat sequences shown in SEQ ID NO: 1; the 5662 bp fragment (the sequence composition shown in SEQ ID NO: 4) contains 3 tandem repeat sequences shown in SEQ ID NO: 1; the 6920 bp fragment (the sequence composition shown in SEQ ID NO: 5) contains 4 tandem repeat sequences shown in SEQ ID NO: 1.

Unless otherwise specified, all the various reagents, materials, etc. used in the following embodiments are commercially available products; unless otherwise specified, all the various testing and detection methods used in the following embodiments are conventional methods in the field, obtainable from textbooks, reference books, or academic journals.

In the present invention, the “deepness and lightness” of watermelon flesh color is detected and defined in the following way:

A CR-410 color difference meter produced by Konica Minolta is used to detect the watermelon flesh color. The measurement results of this instrument consist of 5 data indicators: “L” represents the deepness of the object: 0-100 indicates from black to white; “a” represents the red-green color of the object: a positive value indicates red, and a negative value indicates green; “b” represents the yellow-blue color of the object: a positive value indicates yellow, and a negative value indicates blue; “C” represents the color saturation; “h” represents the hue angle. As the invention focuses on distinguishing intensity within the same base color (most watermelon flesh colors fall into red or yellow). Therefore, the measured “C” value is used to measure the shade of watermelon flesh color; the reading “C” represents color saturation. The judgment indicators for watermelon flesh color intensity are as follows: when “C”>30, the watermelon flesh color is defined as “deep”; when the “C” value is between 25-30 (25≤″C″≤30), the watermelon flesh color is defined as an medium color; when “C”<25, the watermelon flesh color is defined as “light”.

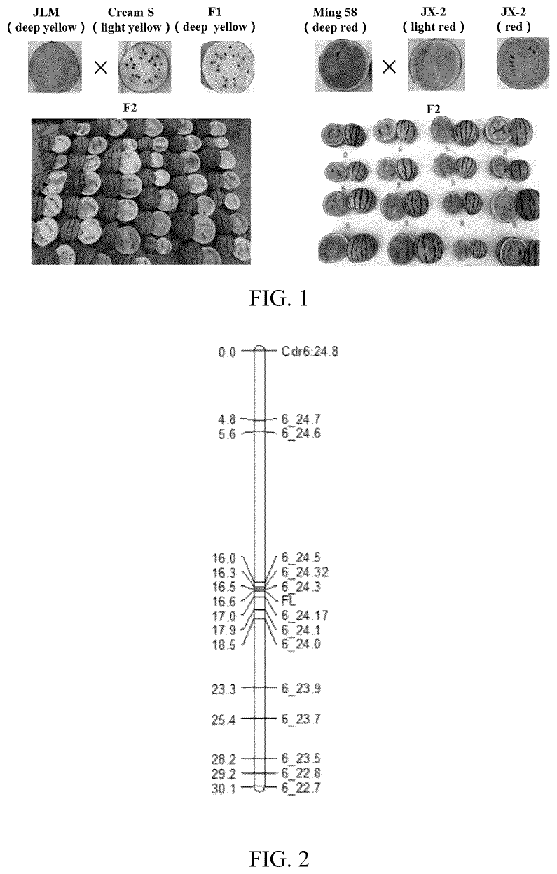

In the following embodiments, among the tested materials used (the cross-section of representative fruits is shown in FIG. 1): Separation population of light and deep red fleshed watermelons: The male parent is JX-2 (light red flesh, “C”=21, Light), and the female parent is Ming 58 (deep red flesh, “C”=33, Deep); as well as the F1 and F2 generations obtained by crossing the two.

Separation population of light and deep yellow fleshed watermelons: The male parent is Cream of Saskatchewan (Cream S, light yellow flesh, “C”=16, Light), and the female parent is JLM (deep yellow flesh, “C”=34, Deep); as well as the F1 and F2 generations obtained by crossing the two.

All test materials are preserved in the Watermelon Germplasm Resource Bank of the Vegetable Research Institute of the Beijing Academy of Agriculture and Forestry Sciences. Anyone can freely obtain relevant materials from there for the purpose of realizing the present invention. The contact address is: Vegetable Research Institute, Beijing Academy of Agriculture and Forestry Sciences, Banjing Rd, Western Suburb, Haidian District, Beijing. Zip code: 100097. Contact person: Zhang Jie. Contact phone number: +8601051503039.

Extraction of genomic DNA from the above test materials: Refer to the method of Murray et al. (1980) (Murray M, Thompson W F. Rapid isolation of high molecular weight plant DNA [J]. Nucl Acid Res, 1980, 8:668-673).

The total RNA of the above test materials was extracted using the EASY spin Plus Plant RNA Kit (Beijing Aidlab Biotech Co., Ltd.), And according to the instructions of the kit, the total RNA of organs such as roots, stems, leaves, flowers, and fruits of the above test materials was extracted respectively. The concentration of the total RNA extracted from the test materials was measured using an ultraviolet spectrophotometer (Shimadzu UV-1201, Japan) based on the OD260 value. Then, the extraction quality of the total RNA from the test materials was detected by 1.2% agarose gel electrophoresis. The first-strand cDNA was synthesized using the Reverse Transcriptase M-MLV (RNase H) reverse transcription kit purchased from TAKARA Company.

### Example 1

This embodiment illustrates the genomic mapping and gene cloning of the watermelon flesh color intensity control gene CIFCI, the discovery of the tandem repeat sequence in its promoter region, and its relationship with flesh color intensity.

### 1. Mapping of CIFCI Gene:

Based on the resequencing data from four watermelon parental lines (JX-2, Ming 58, Cream S, JLM), SNP/InDel sites between parental pairs were identified. Specific high-throughput KASP primers were designed for mapping.

Bulk Segregant Analysis (BSA) was used for parental and DNA pool amplification to screen for polymorphic markers linked to traits, and obtain the initial mapping interval. Thus, the CIFCI gene was mapped to a 3.2 Mb region (22,400,000 to 24,600,000 bp) on chromosome 6 of the watermelon 97103 v2.5 genome.

Then, using the watermelon genome resequencing data, polymorphic markers within this interval were further designed. KASP marker detection was performed using genomic DNA from the test materials to construct a genetic map. Through 14 pairs of 14 KASP markers within the interval (primer compositions in Table 1), the FCI locus was fine-mapped between marker 6-24.17 (position 24.17 Mb on Chr6) and marker 6-24.3 (position 24.3 Mb on Chr6). This interval contained 15 genes, including Cla97C06G121890. The fine mapping map of the constructed watermelon flesh color intensity control gene (i.e., CIFCI gene) is shown in FIG. 2.

TABLE 1
Composition of KASP primer

| SNP_ | | | |
|---|---|---|---|
| ID | primer X | primer _Y | primer _C |
| 6_22. | GAAGGTGACCAAGTT | GAAGGTCGGAGTCAA | GCTGAATTTTTCTGG |
| 7M | CATGCTAAGCCATCG | CGGATTAAGCCATCG | GGTTCGTCTGTT |
| | AATTCGACACTCAAA | AATTCGACACTCAAA | |
| | AT | AA | |
| 6_22. | GAAGGTGACCAAGTT | GAAGGTCGGAGTCAA | GCATCACTAACATTT |
| 8M | CATGCTAATCGTGTG | CGGATTCGTGTGAAA | GTCATTTGTCGAATA |
| | AAACTTACACTCATT | CTTACACTCATTTAA | ATTA |
| | TAACA | CG | |
| 6_23. | GAAGGTGACCAAGTT | GAAGGTCGGAGTCAA | GTGCTACTCCACTCA |
| 5M | CATGCTTATTGCATT | CGGATTCTATTGCAT | TAACCAATTAGTTTT |
| | AGGGATTTCAACCCT | TAGGGATTTCAACCC | |
| | TAC | TTAT | |
| 6_23. | TAGTCCTCATTCGTG | GTCCTCATTCGTGAA | GAATTTCTACCATAT |
| 7M | AATTTTAAATTTTGT | TTTTAAATTTTGC | AGACTATATAATTAT |
| | | | TAATTTTAAAATAT |
| 6_23. | GAAGGTGACCAAGTT | GAAGGTCGGAGTCAA | AGGGTGTGATTTCAT |
| 9M | CATGCTTTTATACAA | CGGATTATTTATACA | TAAATACACCATTAA |
| | GACTCCATGAATAAT | AGACTCCATGAATAA | G |
| | CTCAG | TCTCAT | |
| 6_24. | GCAACCCCTCCCAAC | CAACCCCTCCCAACC | CATATCGGTCCCTTT |
| 0M | CATCTT | ATCTC | ATCAATTCTTTCC |
| 6_24. | TTAAAGAATACACAA | GAAGGTGACCAAGTT | GAAGGTCGGAGTCAA |
| 1M | AACTGAATAGTAGCT | CATGCTGTCATTGGC | CGGATTGTCATTGGC |
| | GTCAGTACTTTTCTT | ATTGCTCCTCCTC | ATTGCTCCTCCTT |
| | CATTTAGAAACCAAA | | |
| | AGAGACATATAAAGA | | |
| | [G/A]AGGAGGAGCA | | |
| | ATGCCAATGACCATA | | |
| | ACTAAGAGAACAAAA | | |
| | CAAATGAGAGAAAAT | | |
| | GCATATCAATCAAAT | | |
| | TACCC | | |
| 6_24. | AAGGGTTGAGTGTTA | GAAGGTGACCAAGTT | GAAGGTCGGAGTCAA |
| 17M | AGCTTCTCGGGAGTC | CATGCTGTAATTCCA | CGGATTGTAATTCCA |
| | ACCGTAATTATGTTG | GATAAGTATTTTATT | GATAAGTATTTTATT |
| | TAATTCCAGATAAGT | GTAATTGC(SEQ ID | GTAATTGG |
| | ATTTTATTGTAATTG | NO: 31) | |
| | [C/G]TTGACATTAT | | |
| | TAATAGTGAAGTTAT | | |
| | ATTTCCTAGATTGCT | | |
| | GCTCCCTAGACATAG | | |
| | GTGATATTATTGCAC | | |
| | CGAAC | | |
| 6_24. | GAGTTAAACAGAAGC | GAAGGTGACCAAGTT | GAAGGTCGGAGTCAA |
| 30M | AAAATATTGAAAATG | CATGCTGTAGCTTTA | CGGATTGGTAGCTTT |
| | AAAATTAGGTTACCT | ATGCTTGGCTTCCC | AATGCTTGGCTTCCT |
| | TGAAAGAAGGGAGGG | | |
| | TAAATGAAGGTGGGG | | |
| | [G/A]GGAAGCCAAG | | |
| | CATTAAAGCTACCAA | | |
| | AGCAACACAACATTA | | |
| | TTTTAACCCTACAAT | | |
| | TCCTTCATTCAAATG | | |
| | TCGTT | | |
| 6_24. | AAATTAGAGCCTCAA | GAAGGTGACCAAGTT | GAAGGTCGGAGTCAA |
| 32M | TTTGGCACCTTATAA | CATGCTATGATATTA | CGGATTGATATTAAC |
| | TCTCGAATGCTAGAA | ACGAGAGCCACACCT | GAGAGCCACACCTG |
| | ACAATCATGATATTA | A | |
| | ACGAGAGCCACACCT | | |
| | [A/G]ACTAAAATGA | | |
| | TTCTTGACACTATAA | | |
| | ATAGCCTCATTCAAC | | |
| | AACTCCGAATGAGAG | | |
| | AGTAAGTAACTTTCA | | |
| | AAAGA | | |
| 6_24. | GAAGGTGACCAAGTT | GAAGGTCGGAGTCAA | GTTTTGTATTGGGGA |
| 5M | CATGCTCCTCCATCC | CGGATTACCTCCATC | TTTGCGGTATAGAAA |
| | AAATGGCCTTCTC | CAAATGGCCTTCTA | |
| 6_24. | GAAGGTGACCAAGTT | GAAGGTCGGAGTCAA | ATTTGTTTGAGATTA |
| 6M | CATGCTTAAGTTGGC | CGGATTTATTAAGTT | TCCTTTTATCTACAT |
| | AAGCTTAGGCCGC | GGCAAGCTTAGGCCG | GGAT |
| | | A | |
| 6_24. | GAAGGTGACCAAGTT | GAAGGTCGGAGTCAA | CAGAGTTCGAATCTT |
| 7M | CATGCTCCAATCTTT | CGGATTCAATCTTTG | TGTGAACATACGAAA |
| | GGTATTTCAACTGCA | GTATTTCAACTGCAA | |
| | ACA | CG | |
| 6_24. | GAAGGTGACCAAGTT | GAAGGTCGGAGTCAA | GTTTTTGACGCATGG |
| 8M | CATGCTCCTCCTCCA | CGGATTCTCCTCCAA | CGGTTGGAATT |
| | ACGCTTTCCAACA | CGCTTTCCAACC | |

2. Cloning of Candidate Genes, Discovery of Tandem Repeat Sequences in the Promoter Region, and their Relationship with Watermelon Flesh Color Intensity.

Based on the genome annotation results, after analyzing the sequence information of 15 candidate genes in the initial mapping interval of the gene locus controlling watermelon flesh color intensity using resequencing data, the following primers were designed:

The specific primer (FCI primer) for amplification of the CDS sequence of the CIFCI gene is as follows:

Upstream sequence:
5′-ATGGCTCCCAAAGCTGGAAAAAC-3;
Downstream sequence:
5′-TCAACTTGAAACCTCAACAATC-3′;

The specific primer (FCI-P) for amplification of the promoter region sequence of the CIFCI gene is as follows:

Upstream sequence:
(SEQ ID NO: 6)
5′-CAAGGATAATTTTAAAATAATG-3′;
Downstream sequence:
(SEQ ID NO: 8)
5′-GTAAAGATGGGTTGGGTTGTTTAC-3′;

The specific primer (FCI-SV) for amplification of the SV with differences in the promoter region of the CIFCI gene is as follows:

Upstream sequence:
(SEQ ID NO: 6)
5′-CAAGGATAATTTTAAAATAATG-3′;
Downstream sequence:
(SEQ ID NO: 7)
5′-ATATACTAATATAATTTGTAGGG-3′

Using the genomic DNA of four watermelon parents (JX-2, Ming 58, Cream S, JLM) as templates, PCR amplification was carried out with the above-mentioned FCI primer, FCI-P primer, and FCI-SV primer respectively.

The reaction system (20 μL) for the PCR amplification reaction was as follows: 2 μL of 10×TransStart Taq Buffer containing 15 mM MgCl₂; 0.8 μL of 2.5 mM dNTPs; 0.9 U of TransStart Taq DNA Polymerase; 0.5 μL of 10 mM upstream primer and 0.5 μL of 10 mM downstream primer; 20 ng of template DNA; and ddH₂O to make up to 20 μL.

The PCR amplification reaction program was: Stage 1: Pre-denaturation at 94° C. for 5 min; Stage 2:94° C. for 30 s, 55° C. for 30 s, 72° C. for 2 min 30 s, for a total of 38 cycles; Stage 3: Extension at 72° C. for 10 min; Stage 5: Storage at 4° C.

The results showed that the full-length fragment obtained by amplification with the above FCI primer was 5547 bp. After sequencing, it was found to be exactly the CDS of the Cla97C06G121890 gene, which was consistent with the sequence composition in the Cucurbit Genomics Database (website: http://cucurbitgenomics.org/v2/feature/gene/Cla97C06G121890).

Using the above FCI-P primer, a promoter sequence with a length of 3146 bp was obtained through amplification in the watermelon parental materials JX-2 and Cream S with light flesh color. The sequencing results showed that the sequence composition was shown in SEQ ID NO: 2. A promoter sequence with a length of 5662 bp was obtained through amplification in the watermelon parental materials Ming 58 and JLM with deep flesh color. The sequencing results showed that the sequence composition was shown in SEQ ID NO: 4. By comparing SEQ ID NO: 2 and SEQ ID NO: 4, it can be found that SEQ ID NO: 2 has 1 tandem repeat sequence shown in SEQ ID NO: 1, while SEQ ID NO: 4 has 3 tandem repeat sequences shown in SEQ ID NO: 1.

Using the above SV identification primer FCI-SV, a fragment with a length of 1343 bp was obtained through amplification in the watermelon parental materials JX-2 and Cream S which both have light flesh color, and a fragment with a length of 3859 bp was obtained through amplification in the watermelon parental materials Ming 58 and JLM which both have deep flesh color. It can be seen that the deep-fleshed parental material is 2516 bp (2*1258 bp) longer than the light-fleshed parental material. After sequencing, it was found that there are large-fragment structural variations (SV) in the deep flesh and light flesh parental materials. Some promoters in the deep-fleshed watermelon material have repeated insertion sequences. Specifically, the 1343 bp fragment amplified from the light-fleshed parental material contains a tandem repeat sequence shown in SEQ ID NO: 1, and the 3859-bp fragment amplified from the deep-fleshed parental material contains 3 tandem repeat sequences shown in SEQ ID NO: 1. The difference of 2516 bp in length is exactly due to the difference of 2 tandem repeat sequences which is 1258 bp shown in SEQ ID NO: 1. The results of this amplification are consistent with the results of bioinformatics analysis.

### Example 2

This embodiment is used to illustrate the application of the tandem repeat sequence in the promoter region of the CIFCI gene in identifying watermelon flesh color intensity.

18 watermelon materials (Ming 58, AU, ZHT, L600, XHB, TS409, Sanbai, k Wushuang, PI296341, Improved TWF, Hard-fleshed TWF, GS12, GS10, GS89, GS41, QM4K, Congo, 97103) were selected, and genomic DNA was extracted from each. PCR amplification was performed using the following primer (the PCR system and reaction program were the same as those in Example 1):

Upstream primer:
(SEQ ID NO: 6)
5′-CAAGGATAATTTTAAAATAATG-3′;
Downstream primer:
(SEQ ID NO: 7)
5′-ATATACTAATATAATTTGTAGGG-3′

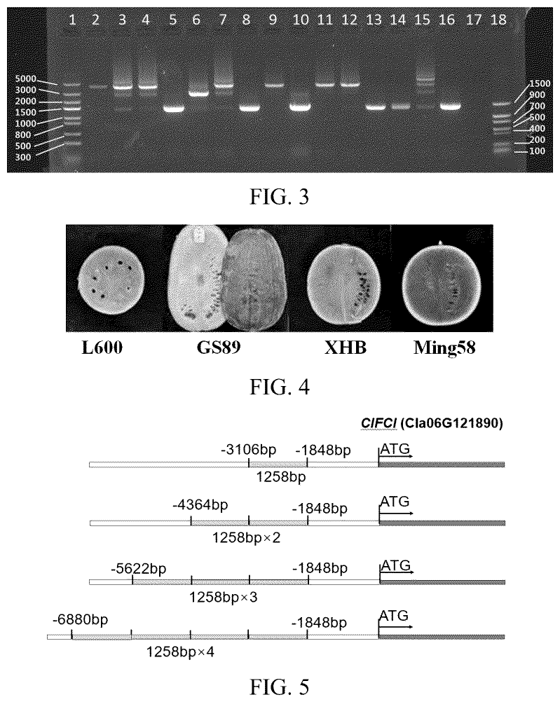

The PCR amplification products were subjected to electrophoresis. The results showed that there were four types of PCR products with lengths of 1343 bp, 2601 bp, 3859 bp, and 5117 bp in the promoter region among different watermelon materials. After sequencing, it was found that all were caused by different numbers of tandem repeats of the 1258-bp sequence shown in SEQ ID NO: 1 (the electrophoresis results are shown in FIG. 3. In the figure, Lane 1 and Lane 18 are both DNA markers (unit: bp), and Lane 17 is a blank control without any DNA template; see Table 2 for flesh color). Meanwhile, a CR-410 color difference meter produced by Konica Minolta was used to detect the flesh color of watermelons, and the “C” values of each tested watermelon material were measured (Table 2).

TABLE 2
Fragment lengths of amplification and flesh
color of 18 different watermelon materials

| Lane in | | PCR product | “C” | Flesh Color |
|---|---|---|---|---|
| FIG. 3 | Watermelon material | (bp) | value | Intensity |
| 2 | Ming 58 | 3859 | 33 | deep |
| 3 | AU | 3859 | 40 | deep |
| 4 | ZHT | 3859 | 34 | deep |
| 5 | L600 | 1343 | 20 | light |
| 6 | XHB | 2601 | 28 | medium |
| 7 | TS409 | 3859 | 31 | deep |
| 8 | San Bai | 1343 | 14 | light |
| 9 | K Wushuang | 3859 | 32 | deep |
| 10 | PI296341 | 1343 | 13 | light |
| 11 | Improved TWF | 3859 | 33 | deep |
| 12 | Hard-fleshed TWF | 3859 | 31 | deep |
| 13 | GS12 | 1343 | 22 | light |
| 14 | GS10 | 1343 | 23 | light |
| 15 | GS89 | 5117 | 37 | deep |
| 16 | GS41 | 1343 | 23 | light |
| — | QM4K | 2601 | 31 | deep |
| — | Congo | 1343 | 23 | light |
| — | 97103 | 1343 | 22 | light |

The electrophoretic fragments of these 18 watermelon materials were recovered and sequencing. The results showed that the sequence compositions of the four types of PCR products of 1343 bp, 2601 bp, 3859 bp, and 5117 bp were as shown from positions 1-1343 of SEQ ID NO: 2, positions 1-2601 of SEQ ID NO: 3, positions 1-3859 of SEQ ID NO: 4, and positions 1-5117 of SEQ ID NO: 5 in the Sequence Listing, respectively. That is, the 1343 bp fragment contained 1 copy of the sequence shown in SEQ ID NO: 1, the 2601 bp fragment contained 2 copies of the sequence shown in SEQ ID NO: 1, the 3859 bp fragment contained 3 copies of the sequence shown in SEQ ID NO: 1, and the 5117 bp fragment contained 4 copies of the sequence shown in SEQ ID NO: 1.

In summary, in the promoter region of the CIFCI gene of 18 watermelon materials, when there is 1 sequence shown in SEQ ID NO: 1, the flesh color is light; when there are 2 sequences shown in SEQ ID NO: 1, the flesh color is medium or deep; when there are 3-4 sequences shown in SEQ ID NO: 1, the flesh color is deep.

Then, four representative materials were further selected: L600, XHB, Ming 58, and GS89 (the flesh color is shown in FIG. 4). PCR amplification was carried out using the following primer (the PCR system and reaction program were the same as those in Example 1):

The upstream sequence is:
(SEQ ID NO: 6)
5′-CAAGGATAATTTTAAAATAATG-3′;
The downstream sequence is:
(SEQ ID NO: 8)
5′-GTAAAGATGGGTTGGGTTGTTTAC-3′.

The PCR amplification products were subjected to electrophoresis, and the amplification products were sequencing. It was found that the sequence compositions of the amplification fragments of L600, XHB, Ming 58, and GS89 were exactly as shown in SEQ ID NO: 2, SEQ ID NO: 3, SEQ ID NO: 4, and SEQ ID NO: 5 in the Sequence Listing, respectively.

Meanwhile, according to the method described in Petry F C and Mercadante A Z. New method for carotenoid extraction and analysis by HPLC-DAD-MS/MS in freeze-dried Citrus and Mango fleshes. Journal of the Brazilian Chemical Society. 2018, 1:205-215, the content of carotenoids (unit: mg/100 gFW, representing the number of milligrams of the target substance per 100 grams of fresh weight) in the flesh of watermelon materials with different flesh colors was detected using Ultra Performance Liquid Chromatography (UPLC) (ExionLC™ AD, https://sciex.com.cn/) and Tandem Mass Spectrometry (MS/MS). The results are shown in Table 3.

TABLE 3
Carotenoid content and colorimeter values in watermelon materials

| | | | | | | | | Colorimeter |
|---|---|---|---|---|---|---|---|---|
| Watermelon | | | | | | | Flesh | Values (L*, a*, |
| material | Lycopene | β-Carotene | α-Carotene | Lutein | Violaxanthin | Neoxanthin | Color | b*, C*, h) |
| L600 | 1.4564 | 0.9526 | 0.0125 | 0.0016 | 0 | 0 | Light | L* 43, a*18, |
| | | | | | | | Red | b*19, |
| | | | | | | | | C* 24, h 39 |
| XHB | 2.9356 | 0.8307 | 0.0322 | 0.0128 | 0 | 0 | Red | L* 36, a*24, |
| | | | | | | | | b*13, |
| | | | | | | | | C* 28, h 29 |
| Ming | 3.7023 | 1.8599 | 0.0304 | 0.0077 | 0.0092 | 0.0062 | Deep | L* 38, a*24, |
| 58 | | | | | | | Red | b*19, |
| | | | | | | | | C* 31, h 38 |
| GS89 | 0 | 2.4557 | 0.0752 | 0.0384 | 0.0171 | 0.0242 | Orange | L* 49, a* 9, |
| | | | | | | | | b*41, |
| | | | | | | | | C* 37, h 76 |

As can be seen from FIG. 4 and Table 3, there is a significant relationship between the number of the sequences shown in SEQ ID NO: 1 in the promoter region of the CIFCI gene in watermelon materials and the depth of the flesh color: when there is only 1 sequence shown in SEQ ID NO: 1, the flesh color is light; when there are 2 sequences shown in SEQ ID NO: 1, the flesh color is medium or deep; when there are 3-4 sequences shown in SEQ ID NO: 1, the flesh color is deep. Moreover, the number of the sequences shown in SEQ ID NO: 1 is positively correlated with the content of carotenoids that cause watermelon flesh color intensity in watermelon materials.

In summary, using the primer of SEQ ID NO: 6 and SEQ ID NO: 7 can detect the SV sequence in the promoter region of the CIFCI gene in watermelon materials with deep and light flesh color, and thereby identify whether the flesh color of the watermelon materials is deep or light. It is also possible to obtain the fragment of the CIFCI gene promoter region in watermelon materials through the amplification of SEQ ID NO: 6 and SEQ ID NO: 8, and then identify whether the flesh color of the watermelon materials is deep or light based on the number of tandem repeat sequences in the obtained fragment of the CIFCI gene promoter region.

### Example 3

This embodiment is used to illustrate the spatiotemporal expression analysis of CIFCI gene, confirming that the SV differences in the promoter region (i.e., the number of tandem repeat sequences in SEQ ID NO: 1) lead to the high expression of the target gene in deep-fleshed varieties.

Fluorescent quantitative PCR was used to detect the expression levels of CIFCI gene in the roots, stems, leaves, flowers, and fruit organs of watermelon variety JLM, and a preliminary analysis of the mechanism of action of CIFCI was conducted. Meanwhile, the cDNAs of the fruits of the two parents, Cream S and JLM, and the representative F2 offspring were analyzed to examine the expression of CIFCI gene in fruits of different flesh color. The details are as follows:

Using the cDNAs of the roots, stems, leaves, flowers, and fruits of the tested materials as templates, PCR amplification was performed with the following fluorescent quantitative PCR primer respectively.

### Fluorescent Quantitative PCR Primer:

FCI_qPCR F:
5′-CAGTAGGTGCATCATCTCCAG-3′;
FCI_qPCR R:
5′-CAATCTCAGCTTCATTGTCGC-3′;
ACTIN F:
5′-CCTACAACTCAATTATGAAGTGTG-3′;
ACTIN R:
5′-GAAATCCACATCTGCTGGAAGGTG-3′.

Among them, primer FCI_qPCR is a specific primer for the spatiotemporal expression analysis of CIFCI, and primer ACTIN is an internal reference primer for fluorescent quantitative PCR.

The reaction system (20 μL) for fluorescent quantitative PCR amplification is as follows: 20 ng cDNA template, 10 μL of 2×GoTaq® qPCR Master Mix, 0.4 μL of 10 μM upstream primer, 0.4 μL of 10 μM downstream primer, and Nuclear-Free Water to make up to 20 μL.

The reaction program for fluorescent quantitative PCR amplification is as follows: Stage 1: Pre-denaturation at 95° C. for 5 min; Stage 2:95° C. for 20 s, 58° C. for 20 s, 72° C. for 30 s, a total of 40 cycles; Stage 3: Release of fluorescence at 94° C. for 8 min.

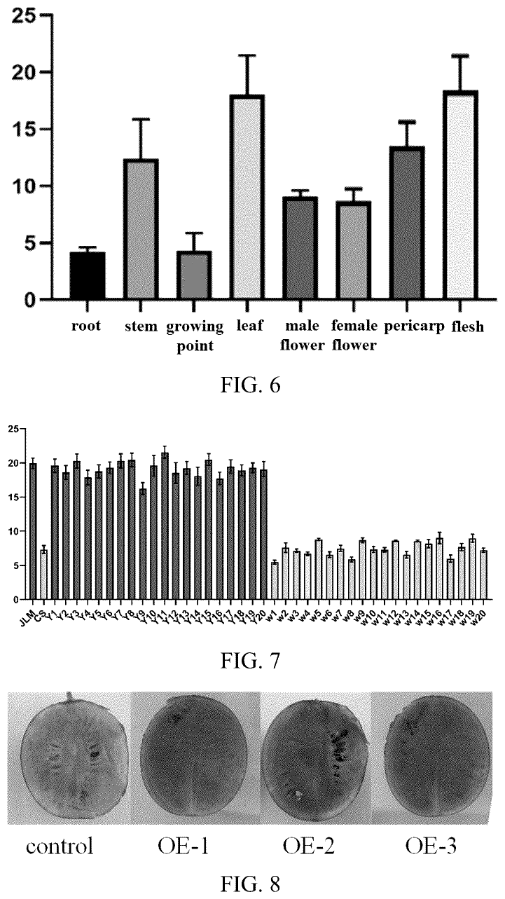

The results are shown in FIG. 6. It can be seen that the CIFCI gene is expressed in the roots, stems, leaves, flowers, and fruits of JLM.

The expression of CIFCI gene in the fruits of 40 F2 generation watermelon materials used for constructing the deep-fleshed and light-fleshed pools sequencing in the parental materials Cream S and JLM and their F2 population was further detected. The results are shown in FIG. 7 (in FIG. 7, Y1-Y20 are representative F2 individual plants with deep yellow flesh, and W1-W20 are representative F2 individual plants with light yellow flesh). The results showed that in different flesh color parents and segregating populations, the expression level of CIFCI gene in the fruits of deep-fleshed materials was significantly higher than that in light-fleshed varieties, with the expression level increased by 2-3 times.

These results indicate that the tandem repeats in the promoter region of CIFCI gene can lead to high expression of CIFCI gene, which deepens watermelon flesh color.

### Example 4

This embodiment is used to illustrate that CIFCI gene overexpression can deepen the flesh color of watermelon.

Construct the overexpression vector of CIFCI gene using pYBA1302 vector: amplification the full-length CDS of CIFCI from the cDNA of watermelon material 97103, and then insert it into the EcoRI/XhoI site of pYBA1302. According to the publicly available watermelon genetic transformation method of our laboratory (Zhang Jie; Guo Shaogui; Ren Yi; Zhang Haiying; Gong Guoyi; Zhou Ming; Wang Guizhang; Zong Mei; He Hongju; Liu Fan; Xu Yong*; High-level expression of a novel chromoplast phosphate transporter CIPHT4; 2 is required for flesh color development in watermelon, New Phytologist, 2017, 213 (3): 1208-1221), transform agrobacterium carrying the target plasmid (FCI-OE) into watermelon material L600. Use specific primer (the upstream primer of the 35S promoter and the downstream primer of the CIFCI gene) and Bar detection test strips to confirm the successful transformation of the overexpression vector into watermelon. After obtaining the homozygous T2 lines, observe the phenotype of the fruit flesh color.

The upstream primer of the 35S promoter and the downstream primer of the CIFCI gene are as follows:

35S-F:
5′-GAAGTTCATTTCATTTGGAGAGG-3′;
CIFCI-R:
5′-ACTTGAAACCTCAACAATCTC-3′.

By constructing transgenic CIFCI gene overexpression lines and observing the phenotype of the flesh color of the offspring (as shown in FIG. 8, where the first “control” is the empty vector control, and the following three are different transgenic lines), it was found that overexpressing the CIFCI gene in the light flesh color variety L600 can deepen the flesh color of the watermelon flesh. Detection showed that the carotenoid content in the flesh increased significantly. This indicates that CIFCI transgenic overexpression can promote the deepening of the flesh color of watermelons.

The above descriptions are only preferred embodiments of the present invention and are not intended to limit the present invention. For those skilled in the art, the present invention may have various modifications and changes. Any modifications, equivalent substitutions, improvements, etc. made within the spirit and principles of the present invention shall be included in the protection scope of the present invention.

1-3. (canceled)

4. A tandem repeat sequence associated with watermelon flesh color intensity control, said tandem repeat sequence is located in the promoter region of watermelon CIFCI gene, having a length of 1258 bp, with a nucleotide sequence composition shown in SEQ ID NO: 1.

5. Application of the tandem repeat sequence in the promoter region of the watermelon CIFCI gene according to claim 4, in identifying watermelon flesh color intensity.

6. A method for identifying watermelon flesh color intensity, said method includes the step of detecting the number of tandem repeat sequences in the promoter region of the CIFCI gene of the watermelon to be tested; wherein, said tandem repeat sequence has a length of 1258 bp, with a nucleotide sequence composition shown in SEQ ID NO: 1; wherein:
When 1 of the tandem repeat sequences is detected, the flesh color of the watermelon to be tested is light;
When 2 of the tandem repeat sequences are detected, the flesh color of the watermelon to be tested is medium or deep;
When 3 or more of the tandem repeat sequences are detected, the flesh color of the watermelon to be tested is deep.

7. The method according to claim 6, characterized in that:
The method for identifying watermelon flesh color intensity includes using the genomic DNA of the watermelon to be tested as a template and performing PCR amplification with the following primer:
The upstream primer shown in SEQ ID NO: 6:
5′-CAAGGATAATTTTAAAATAATG-3′;
The downstream primer shown in SEQ ID NO: 7:
5′-ATATACTAATATAATTTGTAGGG-3′;
If the amplified fragment contains 1 tandem repeat sequence shown in SEQ ID NO: 1, the flesh color of the watermelon to be tested is light;
If the amplified fragment contains 2 tandem repeat sequences shown in SEQ ID NO: 1, the flesh color of the watermelon to be tested is medium or deep;
If the amplified fragment contains 3 or more tandem repeat sequences shown in SEQ ID NO: 1, the flesh color of the watermelon to be tested is deep.

8. The method according to claim 6, characterized in that:
The method for identifying watermelon flesh color intensity includes using the genomic DNA of the watermelon to be tested as a template and performing PCR amplification with the following primer:
The upstream sequence shown in SEQ ID NO: 6:
5′-CAAGGATAATTTTAAAATAATG-3′;
The downstream sequence shown in SEQ ID NO: 8:
5′-GTAAAGATGGGTTGGGTTGTTTAC-3′;
If the amplified fragment contains 1 tandem repeat sequence shown in SEQ ID NO: 1, the flesh color of the watermelon to be tested is light;
If the amplified fragment contains 2 tandem repeat sequences shown in SEQ ID NO: 1, the flesh color of the watermelon to be tested is medium or deep;
If the amplified fragment contains 3 or more tandem repeat sequences shown in SEQ ID NO: 1, the flesh color of the watermelon to be tested is deep.

9. (canceled)
